(19) United States
(12) Patent Application Publication
(10) Pub. No.: US 2018/0192874 A1
(43) Pub. Date: Jul. 12, 2018

(54) BODY PARAMETER MONITORING DEVICE

(71) Applicant: STEADYSENSE GMBH, SEIERSBERG-PIRKA (AT)

(72) Inventors: WERNER KOELE, LIEBOCH (AT); GERALD HOLWEG, GRAZ (AT); CAROLIN KOLLEGGER, STALLHOFEN (AT); JOHANNES SCHWEIGHOFER, GRAZ (AT)

(21) Appl. No.: 15/749,547

(22) PCT Filed: Aug. 4, 2016
(86) PCT No.: PCT/EP2016/001346

§ 371 Date: Feb. 1, 2018

(30) Foreign Application Priority Data
Aug. 4, 2015 (DE) 10 2015 010 189.7

(51) Int. Cl. A61B 5/00; A61B 5/01; A61B 5/053; A61B 5/145; H01Q 9/04; H01Q 1/52; H01Q 23/00; H01Q 1/38

(52) U.S. Cl. CPC A61B 5/002; A61B 2562/029; A61B 5/0531; A61B 5/14539; H01Q 9/0407; H01Q 1/52; H01Q 23/00; H01Q 1/38; A61B 2560/0214; A61B 2560/0412; A61B 2560/0468; A61B 2560/0475; A61B 2562/0247; A61B 2562/0276; A61B 5/01

(57) ABSTRACT

An apparatus for determining at least one physiological parameter includes a carrier and one or more semiconductor components. At least one of the semiconductor components includes a sensor for sensing a physiological parameter. A first antenna may be attached to at least one of the one or more semiconductor components.

### CROSS-REFERENCE TO RELATED APPLICATIONS

This application claims priority to International Application Serial No. PCT/EP2016/001346, filed Aug. 4, 2016, which claims priority to German Application No. 10 2015 010 189.7, filed Aug. 4, 2015. International Application Serial No. PCT/EP2016/001346 is hereby incorporated herein in its entirety for all purposes by this reference.

### FIELD OF THE INVENTION

The present description relates to methods, apparatus and systems for determining or monitoring at least one physiological parameter. The present description relates in particular to methods, apparatus and systems for electronically measuring and monitoring the body temperature of a user.

### BACKGROUND

Monitoring physiological parameters such as the body temperature are important in many medical applications. Standard measurement methods apply. For example, manual thermometers are still frequently used to determine the body temperature at specific locations of the body at certain points in time. These traditional thermometers provide one-time measurements which are only applied on an irregular basis. In addition, the reliability of these conventional measurements depends on the correct use of the apparatus and often leads to wrong results.

### SUMMARY OF EMBODIMENTS OF THE INVENTION

The present invention suggests a method, an apparatus and system according to the independent claims. Specific examples and embodiments are defined by the dependent claims.

The present disclosure describes an apparatus for determining at least one physiological parameter. In one aspect, the apparatus comprises a carrier or support and one or more semiconductor components comprising at least one sensor for sensing a physiological parameter. A first antenna may be attached to or in at least one of the one or more semiconductor components. A booster antenna may be attached to the carrier or support, wherein the booster antenna is galvanically isolated from the first antenna.

In a second aspect, the apparatus comprises a carrier or support with a first side and a second side, at least one semiconductor component attached to the first side and at least one sensor electrode attached to the second side. The at least one sensor electrode may be adapted to come into direct contact with the skin of a user.

### BRIEF DESCRIPTION OF THE DRAWINGS

FIG. 1 shows an example of a sensor tag in an adhesive tape;

FIG. 2A shows the sensor tag with the adhesive tape of FIG. 1 in more detail and FIGS. 2b & 2c show the sensor tag in more detail;

FIG. 3 shows a more detailed example of a disk-like support from different perspectives;

FIG. 4 shows a further example of a disk-like support similar to that in FIG. 3 with an additional sensor;

FIG. 5 shows an example of a sensor tag with an integrated power supply.

### DETAILED DESCRIPTION OF EXEMPLARY EMBODIMENTS OF THE INVENTION

Examples of the present disclosure will now be described in more detail and with respect to the accompanying Figures. The invention as defined by the claims is, however, not limited by the Figures or the examples. Features of one example may be freely combined with features of other examples or features may be omitted unless explicitly stated otherwise.

Identical or similar reference numbers are used to identify same or similar elements or features in the Figures and the description. Description of identical or similar features shown and described with respect to one Figure may be omitted with respect to other figures if these features are identical and/or have the same function. Figures are not necessarily to scale. It is rather the intent to explain the concept.

The present disclosure describes an apparatus for determining at least one physiological parameter. The physiological parameter may be a body temperature, a humidity value or the presence, absence or amount of a specific compound or group of compounds.

In one aspect, the apparatus comprises a carrier or support and one or more semiconductor components comprising at least one sensor for sensing a physiological parameter. A first antenna may be attached to in at least one of the one or more semiconductor components, A second antenna may be attached to the carrier, wherein the second antenna is galvanically isolated from the first antenna. The second antenna may be a booster antenna.

The semiconductor component may be attached to an adhesive tape directly or by means of a support. The apparatus with the semiconductor chip can be attached to the skin of a user or patient with this adhesive tape.

The first antenna and/or the booster antenna may be adapted for one RF transmission standard or may be adapted for a plurality of RF transmission standards.

The booster antenna may be attached to the adhesive tape. For example, the booster antenna may be printed on, woven in, glued to or otherwise fixed to the adhesive tape. Using a booster antenna, no galvanic connection between the booster antenna and the semiconductor chip is required.

If an additional support or carrier substrate is used, the booster antenna may be at least partially implemented on the carrier substrate. The carrier substrate or support may be a foil, disc or a board, for example made from a polymer material or PCB.

In another aspect, the apparatus comprises a carrier or support with a first side and a second side, at least one semiconductor component attached to the first side and at least one sensor electrode attached to the second side. The at least one sensor electrode may be adapted to come into direct contact with the skin of a user.

The sensor electrode and/or the carrier may or may not comprise a protective layer. The protective layer may be made from a hydrophilic material. In other examples it may advantageous to use a hydrophobic material as protection layer. An example for a hydrophobic layer made from a polymeric material may be used. Parylenes are an example of such hydrophobic layers that have good chemical properties and are bio-compatible.

At least one sensor may be a biosensor for sensing a presence or absence of a biological compound. The sensor electrode may for example comprise specific receptors that bind to selected target molecules such as peptides, hormones or proteins. The specific receptor molecules may be designed or selected for the desired target. An impedance sensor may be used to detect the presence or absence and optionally the quantity of the target molecule bound to the receptor molecule at the sensor electrode.

Each sensor tag may have a unique identifier, which is provided with the data that is read out, and allows the data to be associated with a particular sensor, patient, etc.

An antenna element may be attached to the carrier. In particular a booster antenna can be used.

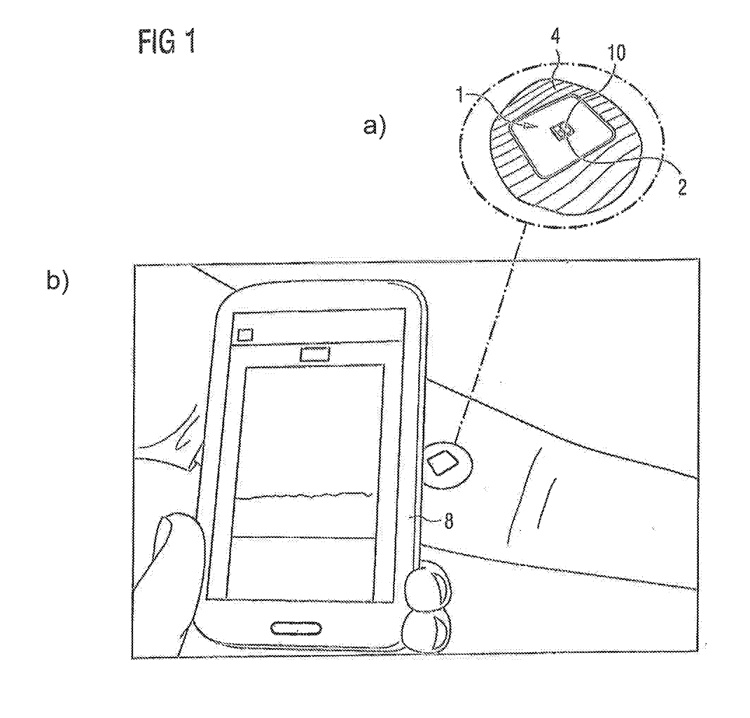

FIGS. 1a and b show an example of a sensor tag 1 attached to an adhesive tape 4 such as a medical tape. The sensor tag 1 comprises at least one semiconductor chip 10 that may be attached to the adhesive tape 4. The semiconductor chip 10 may be directly attached to the adhesive tape 4 or may, as shown in FIG. 1a, be attached to a support 2, which in turn is placed on the adhesive tape 4. In this way the semiconductor chip 10 can be taped to the skin of a user or patient as shown in FIG. 1b. The sensor tag 1 is attached to the adhesive side of the adhesive tape 4 and faces towards the skin when the tape is attached to the skin. The sensor tag 1 may thereby come into direct contact with the skin of the user.

In the example shown in FIG. 1b, the sensor tag 1 is attached to the arm of the user. The arm is chosen for illustrative purposes only. Other places on the body of a user may be more useful to obtain more reliable results.

The at least one semiconductor chip 10 comprises at least one sensor 12 for sensing a physiological parameter such as for example a temperature. The sensor tag 1 may comprise additional electronic elements in the semiconductor chip 10 or elsewhere for data transmission and data storage as will be explained in more detail below. The elements for data transmission may be adapted to use NEC or RFID communication or any other type of radio frequency communication, such as for example the Bluetooth standard, for transmitting data from the sensor tag 1 to a reading device 8 as schematically shown in FIGS. 1a and 1b. Some communication protocols may be problematic in a hospital environment, if the protocol transmits continuously or at regular intervals without an external device initiating the communication. This may interfere with other equipment. A protocol such as NFC is initiated by an external device, so that the sensor tag only communicates after a request has been received (passive protocol).

Another potential issue with communication protocols is the frequency spectrum being used. It may be desirable that the sensor tag use frequencies which do not conflict with other equipment, for example in a hospital environment. The transmission range of the protocol used should also be suited to environments such as a hospital. It may be advantageous to have a transmission range adapted to a usage where a reading device is physically close to the sensor tag, for example 5 cm.

The reading device 8 may be a specific device or may be implemented using a mobile communications device such as a smart phone, a tablet computer with an appropriate software or application thereon or a similar device.

The reading device 8 may be used to read out data collected in the sensor tag 1 and may be optionally used to program the sensor tag 1 if needed. The sensor tag 1 may comprise an energy supply and a recording apparatus that can take measurements even if no reading device is connected to the sensor tag 1 enabling continuous measurements over extended periods such as days, weeks or month.

If the sensor tag 1 is attached to the skin, a continuous measurement of temperatures may be taken. A temperature measurement may be taken every second, every minute or in any other time interval that is useful for the desired measurement. The measured temperature values or other data in relation to the temperature values may be stored in a recording apparatus such as an internal storage inside the semiconductor chip 10. If the reading device 8 is connected to the sensor tag 1, the data stored in the sensor tag 1 can be transmitted to the reading device 8 where they can be displayed, further evaluated or further transmitted. Data can be collected and read without the presence of the reading device 8 and a continuous data series can be collected.

Transmission standards like Bluetooth, NFC or RFID or any other evolving transmission standards can be used for data transmission between the reading device 8 and the sensor tag 1. Useful frequencies can be RFID wireless communication frequencies in the range from 125 kHz up to 5.7 Ghz. In particular NFC at 13.56 MHz may be used, or Bluetooth at 2.4 GHz, which may be particularly interesting for in-vitro diagnostic use.

NFC has become increasingly popular in mobile communication devices such as mobile phones, smart phones and tablet computer enabling the use of these mobile devices together with corresponding application programs.

The reading device 8 may also be used as a programming device for transferring data to the sensor tag 1 and/or for programming the sensor tag 1. This may be used for activating and deactivating the sensor tag 1, for setting measurement parameters and the like.

For example the reading device 8 may start the measurement activities, or the measurement activities may be started by the first access to read out data. The reading device 8 may also set the interval at which measurements are taken. The measurement interval must be chosen in order that measurement values can be stored in available memory during the time between read-out intervals.

It is also possible that a specific programming device is used for restricting access to programming functions and limiting the programming to specifically trained users. The reading device 8 may in this case only allow readout of the data for displaying and or transferring data to a medical caretaker. It is also possible that the reading device allows programming limited functions such as activation, deactivation or the like.

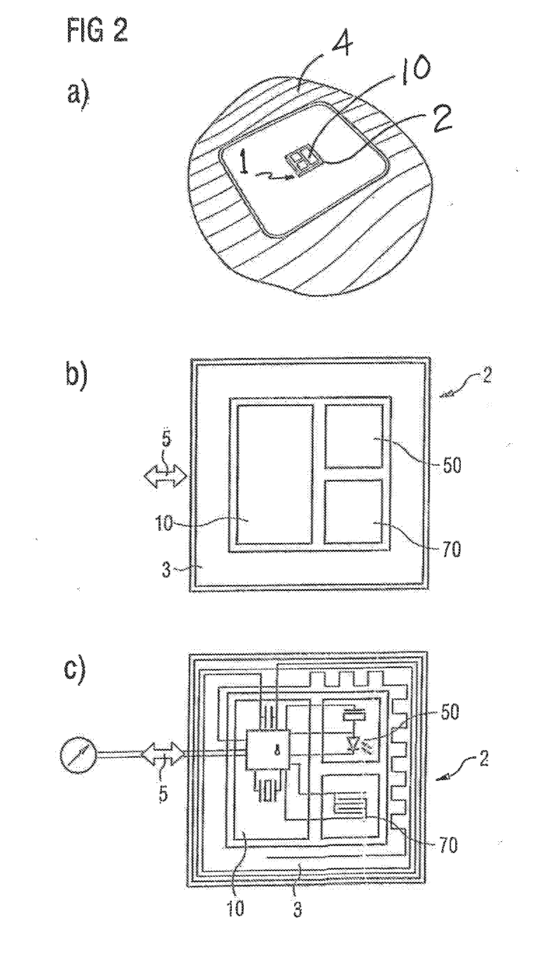

FIG. 2 shows schematic views of the sensor tag 1 of FIG. 1 in more detail. FIG. 2a is an enlargement of the zoomed balloon defined by the chain dashed lines in FIG. 1. In FIG. 2b, the adhesive tape 4 is omitted and only the support 2 with several elements attached to it is shown. The support 2 may be a flexible foil made from a polymeric material as illustrated in FIG. 1. The flexible foil can adapt any curvature of the skin of a user increasing the comfort for a user or patient carrying the sensor tag 1. It may also improve contact of the semiconductor device or of electrodes arranged on the support with the skin of the user or patient. By way of example the support might be a silicone foil, or consist of a flexible foil coated with silicone to ensure biomedical compatibility. Pure silicone (more than 99% silicone) has shown promising results. An alternative might be to use gold or nickel-gold plating or coating.

As an alternative, the support 2 can be a disk or plate made from a more rigid material. Such a disk or plate may provide more stability and may increase reliability of the sensor tag 1 in some applications. The flexibility and rigidity of the material used for the support may be adjusted to the application.

One or more semiconductor chips 10 may be arranged on the support 2. In addition an antenna winding 3 may be printed or otherwise placed on the support 2, for example using an ink-jet printer. The connections may also be achieved this way. The semiconductor die can be attached using either soldering, wire bonding or flip-chip attach. As schematically shown in FIG. 2b, one or more semiconductor chips 10 may comprise an electronic circuit 10, one or more sensors and an electric energy source 50, and optionally one or more sensor electrodes 70. It is possible to use an integrated single semiconductor chip 10 containing all required functions and elements or several semiconductor chips 10 can be used for example different semiconductor dies may be used for sensors and for data collection and data transmission.

A button cell battery may be used as electric energy source 50 and can be attached to the support 2. However, other types of electric energy sources 50 may be used as will be described below.

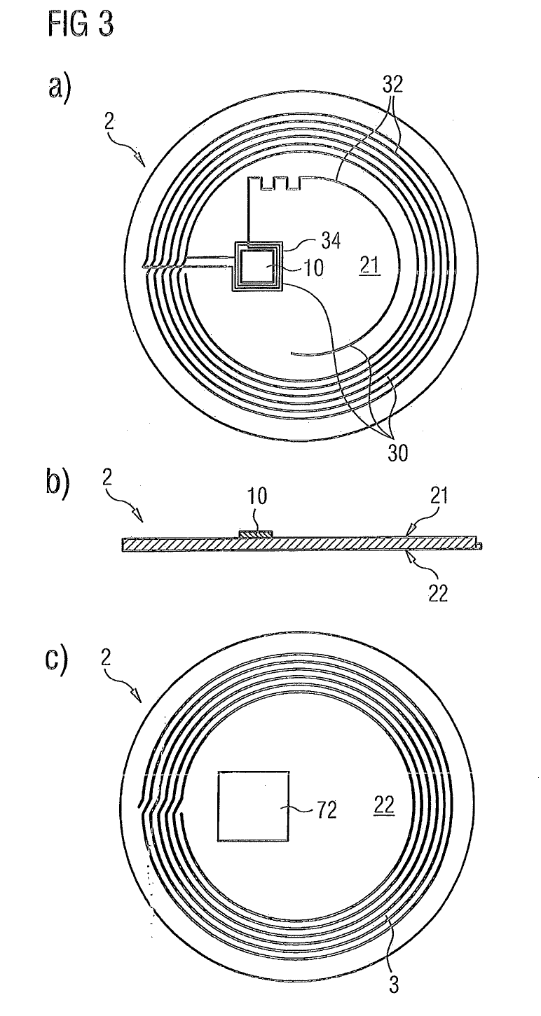

FIG. 3 shows a more detailed example of a sensor tag 1 with a disk-like support 2 from different perspectives with several functional elements arranged on it. The support 2 has a top side 21 and a lower side 22. The lower side 22 may be in use oriented towards the skin or body of the user or patient and may be termed body side or proximal side 22. As schematically shown in FIG. 3c, an electrode or sense pad 72 may be arranged on the lower side 22.

FIG. 3a shows a top view on the top/distal side 21, FIG. 3b a side view and FIG. 3c a top view on the lower/proximal side 22.

The semiconductor chip 10 may be arranged on the top side/distal side 21 as shown in FIGS. 3a and 3b. All electronic circuitry may be arranged inside a single semiconductor chip 10 and the chip may have no external connectors.

As schematically shown in FIG. 3c, the support 2 comprises an antenna element 3. The antenna element 3 can be for example printed on the support 2 or can be integrated in the support 2. As schematically shown in FIG. 3a, the antenna element 3 is used as a booster antenna 30 which is not galvanically connected to the semiconductor element 10. This means that there is no direct conduction path between booster antenna 30 and the semiconductor element 10. The booster antenna 30 is a passive element and has no power supply and no galvanic connection to other elements. As schematically shown in FIG. 3a, the booster antenna 30 comprises a short distance portion 34 disposed relatively closer to the element 10 and at least one long distance portion 32 disposed relatively farther from the element 10.

The at least one long distance portion 32 is adapted for long distance RF (radio frequency) communication with an external device. The long distance portion 32 may be used for transmission standards like NFC and/or RFID or any other type of RF communication. Several types of antenna geometries may be combined as shown in FIG. 3a to allow for communication with different transmission standards. A coating may be provided on the booster antenna 30 to protect the antenna from body liquids or mechanical impact.

The short distance portion 34 is arranged close to the place where the semiconductor chip 10 is placed. For example the short distance portion 34 may be wound around the place where the semiconductor chip 10 is placed as shown in FIG. 3a.

The short distance portion 34 may be used for short distance communication with an internal antenna integrated inside the semiconductor chip 10 and may have working distances of a few millimeters. No galvanic coupling of the semiconductor chip and the antenna is required and no electrical contacts have to be made. This facilitates manufacturing, omits contacting like soldering or bonding steps and positioning of the chip on the support can be less precise. In addition the RF communication is more reliable and mechanically robust compared to an electric connection or bond that can rupture with mechanical load through bending of the support during use.

As schematically shown in FIG. 3c, the support may further comprise an electrode or sense pad 72. In the case of a temperature sensor, the sense pad may be a metal surface or a surface of any other material adapted for good thermal coupling to the body of the user or patient and with good thermal coefficients. In use the sense pad 72 may have the same temperature as the body and may be thermally coupled to the temperature sensor in the semiconductor chip 10. As only thermal coupling is required, no electrical connection has to be established—which increases reliability of the device—and positioning of the chip on the support 2 can be less precise, thus reducing manufacturing efforts.

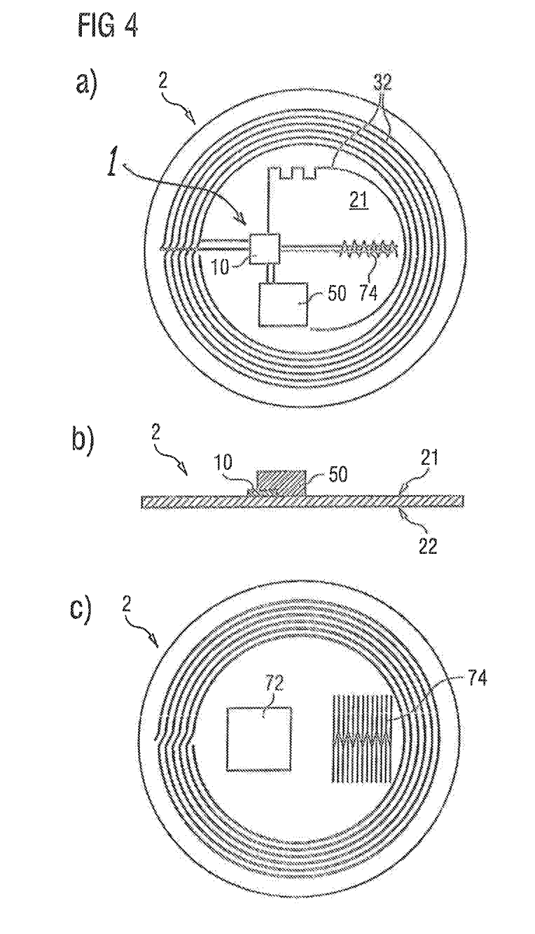

FIG. 4 shows a further example of a sensor tag 1 according to the present disclosure. The support 2 and most other elements are similar or identical to those described with respect to FIG. 3 and the same reference signs are used for similar or identical elements. Description of these features is omitted here and only the differences are described in more detail below.

FIG. 4a shows a top view on the top/distal side 21, FIG. 4b a side view and FIG. 4c a top view on the lower/proximal side 22.

The sensor tag 1 of FIG. 4 has an additional sensor with an additional sensor electrode 74. The additional sensor may be a humidity sensor and may be implemented as an impedance sensor. Thus the additional sensor electrodes 74 may be impedance electrodes. The sensor electrodes may be adapted to come into direct contact with the skin or body of the user or patient. The humidity sensor can be used to determine sweat intensity of the skin and/or can be used to determine wetting of a skin lesion for determining and monitoring healing of this lesion.

Monitoring wound or lesion healing can help reducing the frequency with which band-aids or bandages are replaced thereby reducing the risk of lesion contamination.

The impedance electrodes may be connected to the semiconductor chip 10 and all further elements for sensing humidity may be integrated inside the semiconductor chip.

The sensor tag 1 of FIG. 4 may additionally comprise a power supply 50 such as a button type battery that can be arranged separately on the support 2. The power supply 50 ensures that the semiconductor chip can work even if no external power source is available. This ensures continuous measurements and data storage until read-out.

It may be advantageous to arrange the chip 10 on the opposite side of the support structure 2 from the power supply 50, to ensure better thermocoupling of the chip 10 with the site to be measured. For example, the chip 10 might be on the lower/proximal side 22. Advantageously, the chip 10 may be covered with a coating to ensure biocompatibility, for example with a silicone coating.

In addition, an energy harvester may be used and the battery may be charged, for example via RFID or NFC during data communication with the reading device 8.

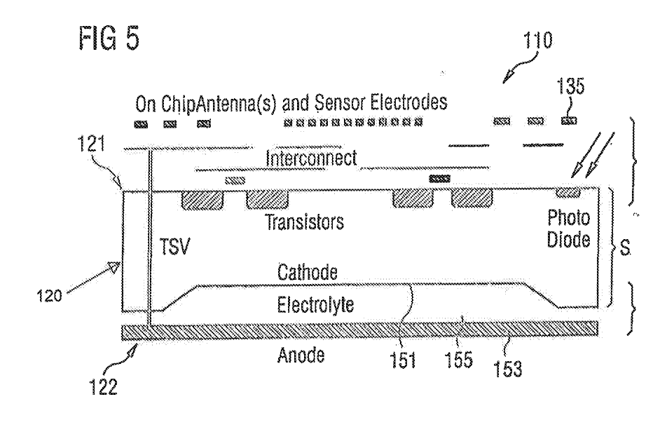

While a separate battery is widely used, other types of batteries may be advantageously used with the present disclosure. FIG. 5 shows a cross section through a semiconductor chip 110 with an integrated battery. The semiconductor chip 110 may have all components as the semiconductor chip 10 described above but with an additional integrated battery. The semiconductor chip 110 has a top side 121 of a semiconductor body 120, at which a plurality of semiconductor elements, such as ASICs, transistors and other components are integrated. A cathode 151 of a battery may be arranged or implemented on the lower side 122 of the semiconductor body 120. The cathode 151 may thus be formed from silicon and may have different structures. A well may be formed to accommodate an electrolyte 155 and a separator. The well may covered by an anode 153. Thus an integrated battery can be used omitting external electrical contacts for the power supply.

An internal antenna element 135 for communication with the booster antenna 30 may arranged on the top side 121 together with other metallization layers used as interconnect layers or sensor elements.

It is to be understood that the examples given above are purely illustrative and a person skilled in the art will combine features shown and explained with one example with other examples. A person skilled in the art will also modify the sensor tag described and will add additional sensors or feature if required.

1. An apparatus for determining at least one physiological parameter, the apparatus comprising:
a first semiconductor component comprising a first sensor for sensing a physiological parameter and data transmission elements for wireless communication;
a first antenna coupled to the first semiconductor component; and
a protective layer in contact with the first sensor, wherein the protective layer ensures biocompatibility of the apparatus and thermal coupling; and
a recording apparatus connected to the first sensor and configured for recording sensor measurement values for subsequent read out.

2. The apparatus of claim 1, wherein the first sensor is selected from the group of a temperature sensor, a humidity sensor, a pH-sensor, a pressure sensor, and an impedance sensor.

3. The apparatus of claim 1, further comprising a wireless communication, apparatus connected to the recording apparatus for communicating wirelessly using a passive protocol.

4. The apparatus of claim 1, wherein the protective layer comprises silicone.

5. The apparatus of claim 1, wherein the protective layer is a silicone foil attached to a support.

6. The apparatus of claim 1, wherein the protective layer is a silicone foil used as a support.

7. The apparatus of claim 1, further comprising a power supply.

8. The apparatus of claim 1, further comprising a booster antenna attached to a first side of a support, wherein the booster antenna is galvanically isolated from the first antenna.

9. The apparatus of claim 8, wherein the support comprises an adhesive tape.

10. The apparatus of claim 9, wherein the booster antenna is attached to the adhesive tape.

11. The apparatus of claim 3, wherein the wireless communication apparatus has a range of up to approximately 5 cm.

12. The apparatus of claim 8, further comprising a sensor electrode attached to a second side of the support opposite to the first side.

13. The apparatus of claim 3, wherein the first semiconductor component comprises the first sensor, a sensor measurement storage and a data transmission element integrated in a single semiconductor die.

14. An apparatus for determining at least one physiological parameter, the apparatus comprising:
a support with a first side and a second side;
a first semiconductor component attached to the first side, and comprising a first sensor for sensing a physiological parameter and data transmission elements for wireless communication;
a first antenna coupled to the first semiconductor component;
a first sensor electrode attached to the second side and adapted to come into direct contact with the skin of a user; and
a recording apparatus for recording sensor measurement values such that they may later be read out.

15. The apparatus of claim 14, wherein the first antenna is attached to the first side of the support.

16. The apparatus of claim 14, wherein the data transmission elements are configured to communicate wirelessly using a passive protocol.
